(19) United States
(12) Patent Application Publication
(10) Pub. No.: US 2014/0046137 A1
(43) Pub. Date: Feb. 13, 2014

(54) Retractor Cover Apparatus and Associated Methods

(71) Applicant: Ronda Duke Brown, Darlington, SC (US)

(72) Inventor: Ronda Duke Brown, Darlington, SC (US)

(21) Appl. No.: 13/569,317

(22) Filed: Aug. 8, 2012

(51) Int. Cl. A61B 1/32

(52) U.S. Cl. 600/203

(57) ABSTRACT

A retractor cover apparatus and associated methods is disclosed. The retractor cover apparatus includes a first sheet having a fluid absorbent material. A second sheet has a medical instrument deflection material. A fastening element is positioned substantially along a periphery of the first sheet and the second sheet, wherein at least a portion of the second sheet is affixed to at least a portion of the first sheet by the fastening element. An unfastened peripheral section of the first sheet and the second sheet form a pocket sized to receive at least a portion of a retractor blade between the first sheet and the second sheet.

### FIELD OF THE DISCLOSURE

The present disclosure is generally related to medical equipment covers and more particularly is related to retractor cover apparatuses and associated methods.

### BACKGROUND OF THE DISCLOSURE

Surgeons require the use of retractors to retract tissue during surgical procedures. As the retractor pulls on the tissue that it contacts, it may place excessive pressure on the tissue. This action may release excess fluids from the tissue. Conventionally, sponges have been used in the wound to absorb the fluids and provide protection from pressure of the retractor blade but the sponges must be repositioned whenever the retractor must be repositioned by the surgeon. However, it is often difficult to appropriately position the sponges proximate to the retractor, or between the retractor and the tissue.

During the procedures the edge of the retractor blade may be used within the close proximity of various medical instruments, which may cause damage to the blade resulting in nicks or gouges in the metal blade. Currently, the retractor blades are not protected from contact with surgical instruments that are placed in the wound. During surgery, when the surgeon is using a sharp instrument or a drill, the sharp edge may come in contact with the retractor resulting in damage to the retractor blade. If the damage is severe, it is possible for metal fragments to gain access to the wound.

Thus, a heretofore unaddressed need exists in the industry to address the aforementioned deficiencies and inadequacies.

### SUMMARY OF THE DISCLOSURE

Embodiments of the present disclosure provide a retractor cover apparatus and associated methods. Briefly described, in architecture, one embodiment of the apparatus, among others, can be implemented as follows. A first sheet has a fluid absorbent material. A second sheet has a medical instrument deflection material. A fastening element is positioned substantially along a periphery of the first sheet and the second sheet, wherein at least a portion of the second sheet is affixed to at least a portion of the first sheet by the fastening element. An unfastened peripheral section of the first sheet and the second sheet form a pocket sized to receive at least a portion of a retractor blade between the first sheet and the second sheet.

The present disclosure can also be viewed as providing methods of using a retractor cover apparatus. In this regard, one embodiment of such a method, among others, can be broadly summarized by the following steps: positioning a retractor blade within a pocket of the retractor cover apparatus, wherein a pocket is formed between a first sheet having a fluid absorbent material and a second sheet having a medical instrument deflection material, wherein at least a portion of the second sheet is affixed to at least a portion of the first sheet with a fastening element; using the retractor blade and retractor cover apparatus within at least one medical operation; contacting the second sheet having the medical instrument deflection material with at least one medical instrument; and deflecting the at least one medical instrument with the medical instrument deflection material, wherein the medical instrument deflection material substantially prevents damage to the retractor blade from contact with the medical instrument.

The present disclosure can also be viewed as providing an elongated retractor sheath. Briefly described, in architecture, one embodiment of the apparatus, among others, can be implemented as follows. The elongated retractor sheath includes a biocompatible fluid absorbent material. A medical instrument deflection material is positioned adjacent to the fluid absorbent material, wherein at least a portion of the fluid absorbent material is affixed to at least a portion of the medical instrument deflection material, and wherein an elongated pocket is created interior of the fluid absorbent material and the medical instrument deflection material. A quantity of stitching is engaged between the fluid absorbent material and the medical instrument deflection material, wherein the quantity of stitching is substantially positioned proximate to at least two edges of the fluid absorbent material and the medical instrument deflection material, and wherein the elongated pocket is formed interiorly of the quantity of stitching.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims.

### BRIEF DESCRIPTION OF THE DRAWINGS

Many aspects of the disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 is a cross-sectional illustration of a retractor cover apparatus, in accordance with a first exemplary embodiment of the present disclosure.

FIG. 2 is a top view illustration of a retractor cover apparatus, in accordance with the first exemplary embodiment of the present disclosure.

FIG. 3 is a cross-sectional illustration of a retractor cover apparatus in use during a medical operation, in accordance with the first exemplary embodiment of the present disclosure.

FIG. 4 is a side cross-sectional illustration of a retractor cover apparatus, in accordance with a second exemplary embodiment of the present disclosure.

FIG. 5 is a top view illustration of a retractor cover apparatus, in accordance with a third exemplary embodiment of the present disclosure.

FIG. 6 is a flowchart illustrating a method of using a retractor cover apparatus in accordance with the first exemplary embodiment of the disclosure.

### DETAILED DESCRIPTION

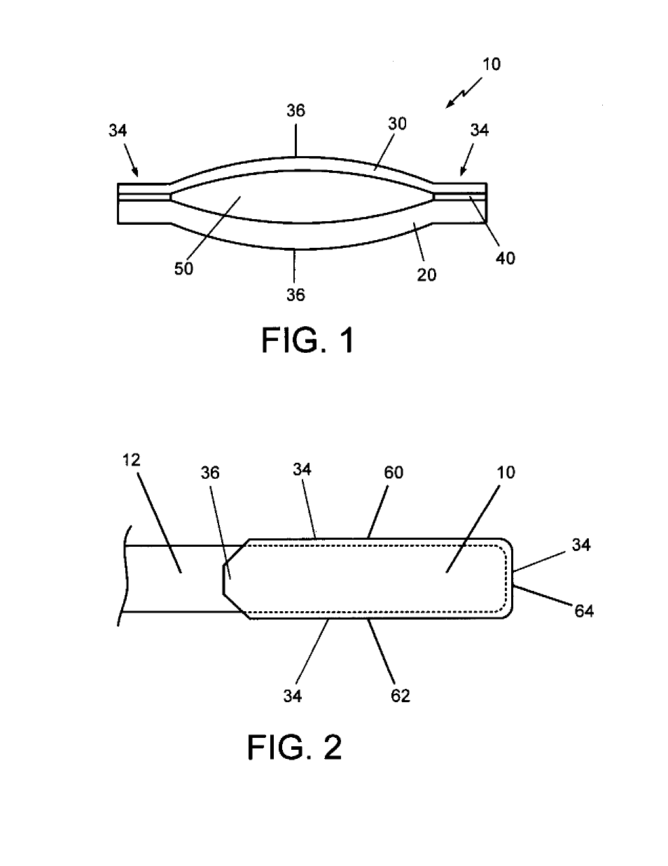

FIG. 1 is a cross-sectional illustration of a retractor cover apparatus 10, in accordance with a first exemplary embodiment of the present disclosure. The retractor cover apparatus 10, which may be referred to herein simply as ‘apparatus 10’ includes a first sheet 20 having a fluid absorbent material. A second sheet 30 has a medical instrument deflection material. A fastening element 40 is positioned substantially along a periphery 34 of the first sheet 20 and the second sheet 30, wherein at least a portion of the second sheet 30 is affixed to at least a portion of the first sheet 20 by the fastening element 40. An unfastened peripheral section 36 of the first sheet 20 and the second sheet 30 form a pocket 50 sized to receive at least a portion of a retractor blade between the first sheet 20 and the second sheet 30.

A retractor is a surgical instrument by which a surgeon can either actively separate the edges of a surgical incision or wound, or can hold back underlying organs and tissues, so that body parts under the incision may be accessed. Retractors are each available in many shapes, sizes, and styles, and generally include a curved, hooked, or angled blade that, when in place, maintains the desired position of a given region of tissue. Retractors may also include two or more opposing blades or hooks which are separated via spring, ratchet, worm gear or some other method and pull on opposite sides of a wound. These retractors may include hand-cranked devices such as rib spreaders (also known as thoracic retractors, or distractors) with which surgeons may forcefully drive tissues apart to obtain the exposure. The retractor may be handheld, clamped in situ, or suspended at the end of a robotic arm.

In accordance with this disclosure, the retractor cover apparatus 10 may be used with any type of retractor or retractor blade, spatula blade, or similar medical device. Common retractors and spatula blades that the apparatus 10 may be used with include, but are not limited to, a Davis brain spatula blade, a Bernstein nasal retractor blade, a Deaver retractor blade, an Eastman retractor blade, a Goelet retractor blade, a Graves vaginal speculum blade, a Harrington retractor blade, a Kelly retractor blade, a Parker retractor blade, a Ribbon retractor blade, a Richardson retractor blade, a Richardson-Eastman retractor blade, a Senn retractor blade, a Taylor spinal retractor blade, a Wieder tongue retractor blade, an Army Navy retractor blade, a micro retractor blade, and a silicone brain spatula.

The apparatus 10 may be used in any medical procedure in any medical field, including surgeries, examinations, and operations on any human being or other living being. Commonly, the apparatus 10 may be used in surgeries or operations where usage of a medical instrument may contact the blade of the retractor, such as with operations having small work sites or tissue openings. The apparatus 10 may also provide many benefits in medical operations where the retractor blade presents a substantial force on the tissue or bone structure of the patient.

The first sheet 20 includes, has, or is partially or completely formed from a fluid absorbent material, such as a sponge material commonly used in medical operations. For example, the fluid absorbent material may include a biocompatible rayon, biocompatible polyester, and/or biocompatible cotton, among other materials, such as any combination of polyester, cellulose, and foam. Any of the fluid absorbent material may be woven or non-woven, with any number of plys and fabrics. In construction, it may be desirable for the entire first sheet 20 to be formed from the fluid absorbent material, as is depicted in FIG. 1. However, the first sheet 20 may be constructed from a number of materials that includes the fluid absorbent material, such as when the fluid absorbent material is bonded to supporting layer formed from a separate material.

The second sheet 30 includes, has, or is partially or completely formed from a medical instrument deflection material. The medical instrument deflection material may commonly be constructed from polytetrafluoroethylene, but other deflection-capable materials may also be used. Similar to the first sheet 20, in construction, it may be desirable for the entire second sheet 30 to be formed from the medical instrument deflection material, as is depicted in FIG. 1. However, the second sheet 30 may be constructed from a number of materials that includes the medical instrument deflection material, such as when the medical instrument deflection material is bonded to supporting layer formed from a separate material.

Depending on the materials used to construct the first and second sheets 20, 30 the respective thickness of the first and second sheets 20, 30 may vary. Commonly, the thickness of the first sheet 20 will be greater than the thickness of the second sheet 30, since the fluid absorbent material used, in whole or in part, to form the first sheet 20 is commonly a fibrous material, whereas the medical instrument deflection material is commonly a non-fibrous material. The overall thickness of the apparatus 10 may ideally be as small as possible to provide a workspace or surgical site for the surgeon that is as unencumbered as possible. The length and width of the apparatus 10 and the pocket 50 formed therein may vary greatly and will largely depend on the size of the retractor(s) that the apparatus 10 is used with. The apparatus 10 may be constructed to fit a single, specific retractor blade or to universally fit a plurality of different retractor blades.

At least a portion of the second sheet 30 is affixed to at least a portion of the first sheet 20 with the fastening element 40. As is shown in FIG. 1, the affixed portion of the first and second sheets 20, 30 are located along a periphery 34 of the first sheet 20 and the second sheet 30, as is discussed further with respect to FIG. 2. The fastening element 40 is positioned between the affixed portions of the first sheet 20 and the second sheet 30 to form the pocket 50. The fastening element 40 may include a number of fastening and bonding materials, structures, or systems. For example, the fastening element 40 may be stitching that is engaged between the first and second sheets 20, 30, or an adhesive bonding material, such as glue, as is shown in FIG. 1.

The pocket 50 is sized to receive all or a portion of a retractor blade, such that the portion of the retractor blade that is to be in contact with the biological tissue, or is likely to be contacted by a medical instrument during a medical operation, is completely covered by the apparatus 10. The unfastened peripheral section 36 of the first sheet 20 and the second sheet 30 may from the pocket 50, wherein the pocket 50 begins at the unfastened peripheral section 36 and occupies the interior space between the first sheet 20 and the second sheet 30. In this configuration, the pocket 50 sized to receive at least a portion of a retractor blade between the first sheet 20 and the second sheet 30. The pocket 50 may be defined by the space created within the apparatus 10 between the first sheet 20, the second sheet 30, and the fastening element 40.

FIG. 2 is a top view illustration of a retractor cover apparatus 10, in accordance with the first exemplary embodiment of the present disclosure. As can be seen in FIG. 2, the apparatus 10 may receive a retractor blade 12 within the pocket (FIG. 1). In FIG. 2, the portion of the retractor blade 12 that is located within the pocket of the apparatus 10 is depicted in broken lines. To accommodate the retractor blade 12 within the apparatus 10, the first and second sheets (FIG. 1) are affixed together in specific places. Notably, the first and second sheets are affixed together around at least a portion of the exterior edge of the apparatus 10, thereby creating the interior space of the pocket between the first and second sheets.

Although the apparatus 10 may have a plurality of edges, it may be common for the apparatus 10 to have three edges—a first edge 60, a second edge 62, and a third edge 64—that proximate to the affixed first and second sheets. These edges 60, 62, and 64 may, in whole or part, be positioned at the periphery 34 of the first and second sheets 20, 30, wherein the unfastened periphery section 36 of the first and second sheets 20, 30 may occupy any portion of the remaining edges of the first and second sheets 20, 30. Thus, the portion of the second sheet that is affixed to the portion of the first sheet may include two distinct elongated edges of the first and second sheets, such as the first and second edges 60, 62. Similarly, the portion of the second sheet that is affixed to the portion of the first sheet may include the two distinct elongated edges, first and second edges 60, 62, as well as one end edge of the first and second sheets, third edge 64. Of course, depending on the shape and design, the apparatus 10 may include any number of edges, wherein any portion of the edges include affixed portions of the first and second sheets, and/or the fastening element (FIG. 1).

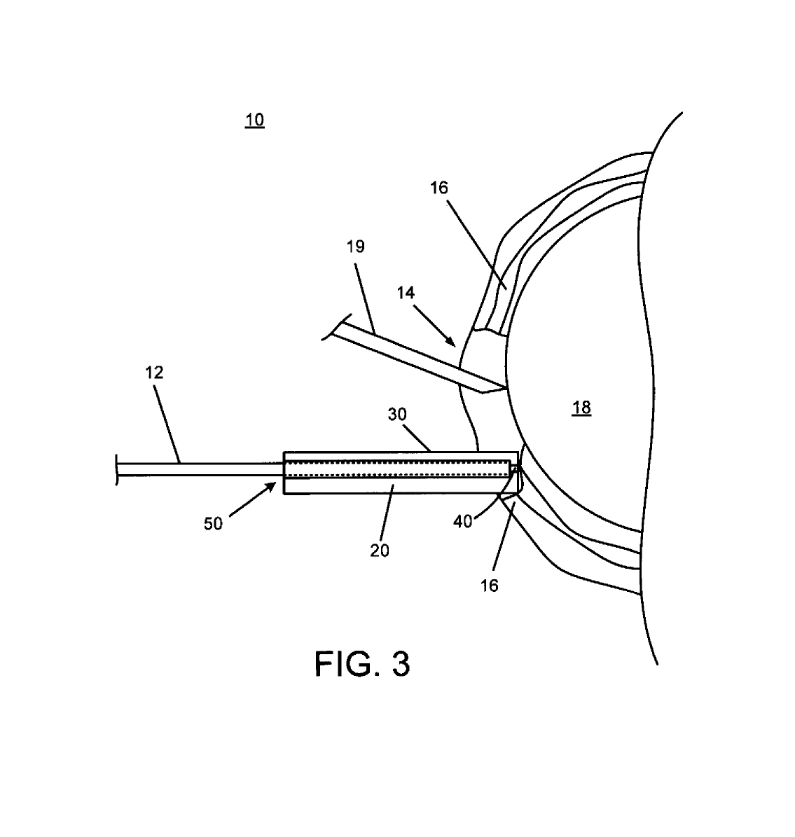

FIG. 3 is a cross-sectional illustration of a retractor cover apparatus 10 in use during a medical operation, in accordance with the first exemplary embodiment of the present disclosure. As is shown in FIG. 3, the apparatus 10 is shown in use with a medical operation. The apparatus 10 is positioned about the retractor blade 12, wherein an end of the apparatus 10 and retractor blade 12 is positioned within a surgical site 14 having a quantity of biological tissue 16 and a quantity of bone 18. The retractor blade 12 with apparatus 10 can be used to bias a portion of the biological tissue 16 away from where a medical instrument 19, such as a drill bit as shown, is being used. The first sheet 20 having the fluid absorbent material may be used to contact the biological tissue, while the second sheet 30 having the medical instrument deflection material may be facing the proximate area where the medical instrument 19 is being used.

Usage of the apparatus 10 may offer many benefits. For example, in the configuration shown in FIG. 3, the positioning of the first sheet 20 allows the fluid absorbent material to absorb fluids from the biological tissue 16, cushion the force of the retractor blade 12 against the biological tissue 16, as well as keep the biological tissue 16 moist when the fluid absorbent material is moist. At the same time, the medical instrument deflection material on the second sheet 30 may provide a deflective barrier if the medical instrument 19 inadvertently contacts the retractor blade 12. The medical instrument defection material of the second sheet 30 may prevent the medical instrument 19 from damaging the retractor blade 12 and possibly removing surface material from the retractor blade 12 that may fall into the surgical site 14.

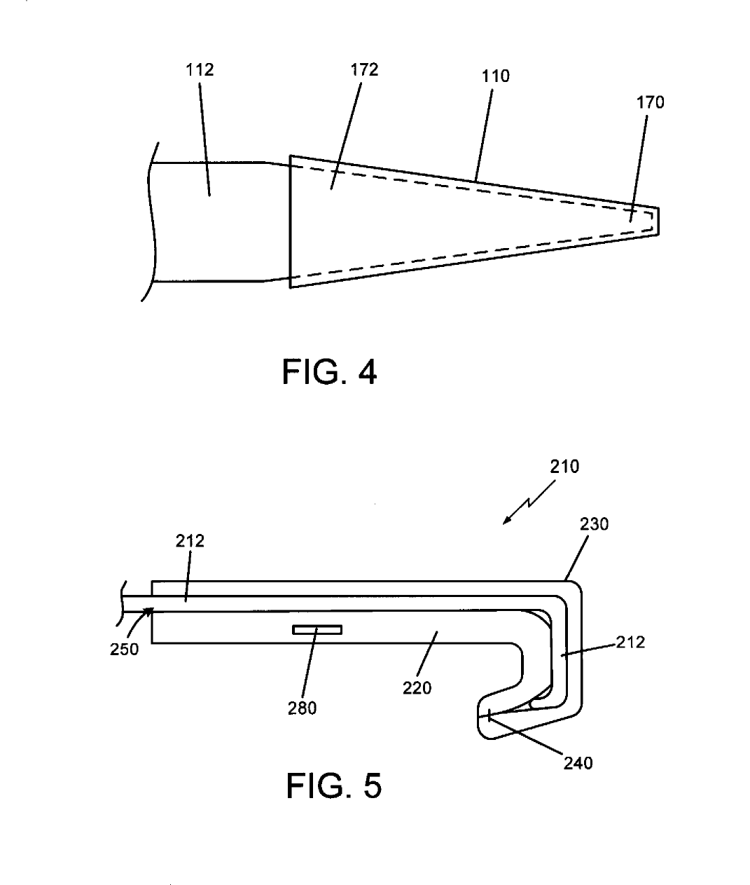

FIG. 4 is a side cross-sectional illustration of a retractor cover apparatus 110, in accordance with a second exemplary embodiment of the present disclosure. The retractor cover apparatus 110, which may be referred to herein simply as ‘apparatus 110’ may be substantially similar to the apparatus 10 of the first exemplary embodiment, in that the apparatus 110 includes a first sheet having a fluid absorbent material and a second sheet having a medical instrument deflection material. At least a portion of the second sheet is affixed to at least a portion of the first sheet, and a fastening element is positioned between the affixed portions of the first sheet and the second sheet wherein a pocket sized to receive at least a portion of a retractor blade is formed between the first sheet and the second sheet. For clarity in disclosure, FIG. 4 does not depict the first and second sheets, the fastening element, or the pocket.

As can be seen in FIG. 4, the apparatus 110 may include a shape that substantially conforms to the shape of a retractor blade 112 (shown partially in broken lines). For example, the retractor blade 112 may have a pointed end 170 that has a narrower width than a middle portion 172 of the retractor blade 112. As the apparatus 110 may be tapered to accommodate a tapered retractor blade 112, the pocket within the apparatus 110 may also be tapered. Thus, the apparatus 110 may be designed to follow the shape of the retractor blade 112 to provide an interface between the retractor blade 112 and the apparatus 110 that minimizes accidental removal of the apparatus 110 from the retractor blade 112, but does not overly encumber the ability to remove the apparatus 110 from the retractor blade 112 when a medical or surgical technician or other operating room staff desires to remove the apparatus 110. The apparatus 110 may be designed to fit with any size or shape of a retractor blade 112.

FIG. 5 is a top view illustration of a retractor cover apparatus 210, in accordance with a third exemplary embodiment of the present disclosure. The retractor cover apparatus 210 of FIG. 5, which may be referred to simply as ‘apparatus 210’ may include any of the features discussed with respect to any other embodiments of the disclosure. For example, the apparatus 210 may include a first sheet 220 having a fluid absorbent material. A second sheet 230 has a medical instrument deflection material, wherein at least a portion of the second sheet 230 is affixed to at least a portion of the first sheet 220. A fastening element 240 is positioned between the affixed portions of the first sheet 220 and the second sheet 230, wherein a pocket 250 sized to receive at least a portion of a retractor blade is formed between the first sheet 220 and the second sheet 230. As is shown in FIG. 5, the fastening element 240 may be a stitched material between the first and second sheets 220, 230.

The apparatus 210 shown in FIG. 5 further includes a radiopaque marker 280 which may be embedded or otherwise affixed to the apparatus 210. Preferably, the radiopaque marker 280 is bonded to or embedded within the first sheet 220. The radiopaque marker 280 may include any material that substantially prevents radiation penetration, such as radiation from X-rays. As one having skill in the art would recognize, this allows the surgeon to account for any apparatus 210 and prevent accidental situations where an apparatus 210 is left within a patient's body after the surgical procedure. Additionally, FIG. 5 depicts the apparatus 210 being used on a retractor blade 212 that is bent or curved. The apparatus 210 may substantially conform to the shape of the retractor blade 212, regardless of the size or shape of any bend or curve that is present within the refractor blade 212.

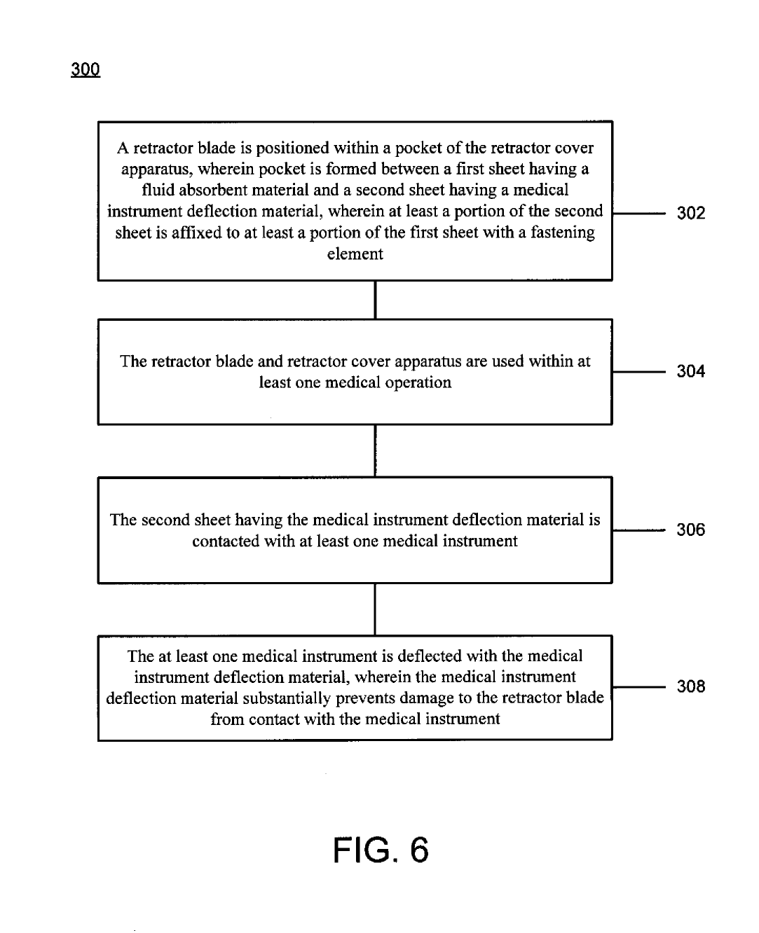

FIG. 6 is a flowchart 300 illustrating a method of using a retractor cover apparatus 10 in accordance with the first exemplary embodiment of the disclosure. It should be noted that any process descriptions or blocks in flow charts should be understood as representing modules, segments, portions of code, or steps that include one or more instructions for implementing specific logical functions in the process, and alternate implementations are included within the scope of the present disclosure in which functions may be executed out of order from that shown or discussed, including substantially concurrently or in reverse order, depending on the functionality involved, as would be understood by those reasonably skilled in the art of the present disclosure.

As is shown by block 302, a retractor blade is positioned within a pocket of the retractor cover apparatus, wherein pocket is formed between a first sheet having a fluid absorbent material and a second sheet having a medical instrument deflection material, wherein at least a portion of the second sheet is affixed to at least a portion of the first sheet with a fastening element. The retractor blade and retractor cover apparatus are used within at least one medical operation (block 304). The second sheet having the medical instrument deflection material is contacted with at least one medical instrument (block 306). The at least one medical instrument is deflected with the medical instrument deflection material, wherein the medical instrument deflection material substantially prevents damage to the retractor blade from contact with the medical instrument (block 308).

The method may further include any number of additional steps or processes, including any of the steps, processes, and functions disclosed with respect to any embodiments of this disclosure. For example, when in use in a medical operation, bodily fluid may be absorbed with the fluid absorbent material of the first sheet. The first sheet may also cushion a force of the retractor blade against a quantity of biological tissue. The fluid absorbent material of the first sheet may be moistened before using the retractor blade and retractor cover apparatus within the at least one medical operation. During or after a medical operation, the retractor cover may be removed from the retractor blade and dispose of accordingly.

It should be emphasized that the above-described embodiments of the present disclosure, particularly, any “preferred” embodiments, are merely possible examples of implementations, merely set forth for a clear understanding of the principles of the disclosure. Many variations and modifications may be made to the above-described embodiments of the disclosure without departing substantially from the spirit and principles of the disclosure. All such modifications and variations are intended to be included herein within the scope of this disclosure and the present disclosure and protected by the following claims.

What is claimed is:

1. A retractor cover apparatus comprising:
a first sheet having a fluid absorbent material;
a second sheet having a medical instrument deflection material;
a fastening element positioned substantially along a periphery of the first sheet and the second sheet, wherein at least a portion of the second sheet is affixed to at least a portion of the first sheet by the fastening element; and
an unfastened peripheral section of the first sheet and the second sheet forming a pocket sized to receive at least a portion of a retractor blade between the first sheet and the second sheet.

2. The retractor cover apparatus of claim 1, wherein the medical instrument deflection material further comprises polytetrafluoroethylene.

3. The retractor cover apparatus of claim 1, wherein the fluid absorbent material further comprises as least one of biocompatible rayon, biocompatible polyester, and biocompatible cotton.

4. The retractor cover apparatus of claim 1, wherein the fastening element further comprises a plurality of stitching engaging the first sheet and the second sheet.

5. The retractor cover apparatus of claim 1, wherein the fastening element further comprises an adhesive bonding material.

6. The retractor cover apparatus of claim 1, further comprising at least one radiopaque marker bonded to the first sheet.

7. The retractor cover apparatus of claim 1, wherein a thickness of the first sheet is greater than a thickness of the second sheet.

8. The retractor cover apparatus of claim 1, wherein the portion of the second sheet affixed to at least the portion of the first sheet further comprises at least two distinct elongated edges of the first and second sheets.

9. The retractor cover apparatus of claim 1, wherein the portion of the second sheet affixed to at least the portion of the first sheet further comprises at least two distinct elongated edges of the first and second sheets and at least one end edge of the first and second sheets.

10. The retractor cover apparatus of claim 1, wherein the shape of the first and second sheets substantially corresponds to a shape of the retractor blade when the retractor blade is positioned within the pocket.

11. The retractor cover apparatus of claim 1, wherein the unfastened peripheral section of the first sheet and the second sheet forming the pocket is tapered.

12. The retractor cover apparatus of claim 1, wherein the unfastened peripheral section of the first sheet and the second sheet forming the pocket is sized to receive at least a portion of at least one of: a Davis brain spatula blade, a Bernstein nasal retractor blade, a Deaver retractor blade, an Eastman retractor blade, a Goelet retractor blade, a Graves vaginal speculum blade, a Harrington retractor blade, a Kelly retractor blade, a Parker retractor blade, a Ribbon refractor blade, a Richardson retractor blade, a Richardson-Eastman retractor blade, a Senn retractor blade, a Taylor spinal retractor blade, a Wieder tongue retractor blade, an Army Navy retractor blade, a micro retractor blade, and a silicone brain spatula.

13. A method of using a retractor cover apparatus comprising the steps of:
positioning a refractor blade within a pocket of the retractor cover apparatus, wherein pocket is formed between a first sheet having a fluid absorbent material and a second sheet having a medical instrument deflection material, wherein at least a portion of the second sheet is affixed to at least a portion of the first sheet with a fastening element;
using the retractor blade and refractor cover apparatus within at least one medical operation;
contacting the second sheet having the medical instrument deflection material with at least one medical instrument; and
deflecting the at least one medical instrument with the medical instrument deflection material, wherein the medical instrument deflection material substantially prevents damage to the retractor blade from contact with the medical instrument.

14. The method of claim 13, further comprising the step of absorbing bodily fluid with the fluid absorbent material of the first sheet while using the retractor blade and retractor cover apparatus within the at least one medical operation.

15. The method of claim 13, further comprising the step of cushioning a force of the retractor blade against a quantity of biological tissue with the first sheet.

16. The method of claim 13, further comprising the step of moistening the fluid absorbent material of the first sheet before using the retractor blade and retractor cover apparatus within the at least one medical operation.

17. The method of claim 13, wherein the step of deflecting the at least one medical instrument with the medical instrument deflection material further comprises deflecting at least one medical drill bit.

18. The method of claim 13, wherein the step of deflecting the at least one medical instrument with the medical instrument deflection material further comprises preventing surface damage to the retractor blade.

19. The method of claim 13, further comprising the step of removing the retractor cover apparatus from the retractor blade.

20. An elongated retractor sheath comprising:
a biocompatible fluid absorbent material;
a medical instrument deflection material positioned adjacent to the fluid absorbent material, wherein at least a portion of the fluid absorbent material is affixed to at least a portion of the medical instrument deflection material, and wherein an elongated pocket is created interior of the fluid absorbent material and the medical instrument deflection material; and
a quantity of stitching engaged between the fluid absorbent material and the medical instrument deflection material, wherein the quantity of stitching is substantially positioned proximate to at least two edges of the fluid absorbent material and the medical instrument deflection material, and wherein the elongated pocket is formed interiorly of the quantity of stitching.
